(19) United States
(12) Patent Application Publication
(10) Pub. No.: US 2010/0158999 A1
(43) Pub. Date: Jun. 24, 2010

(54) COMBINATION OF TRIAZINE DERIVATIVES AND HMG-COA REDUCTASE INHIBITORS

(76) Inventors: Gérard Moinet, Orsay (FR); Daniel Cravo, Sartrouville (FR); Didier Mesangeau, Combs La Ville (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/160,504

(22) PCT Filed: Dec. 18, 2006
(86) PCT No.: PCT/EP2006/012184

§ 371 Date: Jul. 10, 2008

(30) Foreign Application Priority Data
Jan. 13, 2006 (FR) 06/00343

(51) Int. Cl. A61K 9/48; A61K 31/53; A61K 31/58; A61K 9/28; A61P 3/04; A61P 3/10; A61P 7/12; A61P 9/10; A61P 9/00

(52) U.S. Cl. 424/456; 514/245; 514/171; 424/474

(57) ABSTRACT

The present patent application relates to combinations of a triazine derivative with an HMG-CoA reductase inhibitor.

### FIELD OF THE INVENTION

The present invention relates to a pharmaceutical composition of triazine derivatives or described pharmaceutically acceptable salts thereof with an HMG-CoA reductase inhibitor, for the manufacture of a medicament that can be used in the treatment of non-insulin-dependent diabetes and pathologies associated with insulin resistance syndrome.

### TECHNICAL BACKGROUND

“Diabetes mellitus” (or diabetes) is one of the most prevalent diseases in the world today. Individuals suffering from diabetes have been divided into two classes, namely type I or insulin-dependent diabetes mellitus and type II or non-insulin-dependent diabetes mellitus (NIDDM). Non-insulin-dependent diabetes mellitus (NIDDM) accounts for approximately 90% of all diabetics, and is estimated to affect 12 to 14 million adults in the United States alone (6.6% of the population). NIDDM is characterised both by fasting hyperglycaemia and exaggerated postprandial increases in plasmatic glucose levels. NIDDM is associated with a variety of long-term complications, including microvascular diseases, such as retinopathy, nephropathy and neuropathy, and macrovascular diseases, such as coronary heart disease. Numerous studies in animal models show a causal relationship between long-term complications and hyperglycaemia. Recent results obtained by the Diabetes Control and Complications Trial (DCCT) and the Stockholm Prospective Study have for the first time demonstrated this relationship in man by showing that insulin-dependent diabetics have a substantially lower risk of development and progression of these complications if they are subjected to tighter glycaemic control. Tighter control is also expected to benefit NIDDM patients.

Hyperglycaemia in the case of NIDDM is associated with two biochemical anomalies, namely insulin resistance and insufficiency of insulin secretion.

The initial treatment of NIDDM is based on a controlled diet and controlled physical exercise, since a considerable number of diabetics are over-weight or obese (˜67%) and since loss of weight can improve insulin secretion and sensitivity to insulin and lead to normal glycaemia.

Patients suffering from a hyperglycaemia that cannot be controlled solely by diet and/or physical exercise are then treated with oral antidiabetics.

A number of categories of oral antidiabetics are currently used in monotherapy for the treatment of NIDDM:
insulin secretion stimulators. They are represented, firstly, by sulfonylureas (SU) and by “glinides”. As regards SUs, mention will be made in particular of carbutamide (Glucidoral®), glibenclamide/glyburide (Daonil®, Euglucan®), glibomuride (Glutril®), gliclazide (Diamicron®), glimepiride (Amarel®) and glipizide (Glibenese®). As regards the “glinides”, mention will be made in particular of repaglinide (NovoNorm®);
agents that reduce glucogenesis, represented by the biguanides. Mention will be made in particular of metformin (Glucophage®, Stagid®);
insulin sensitisers, represented mainly by thiazolidinediones (TZD). Mention will be made in particular of pioglitazone (Actos®) and rosiglitazone (Avandia®);
alpha-glucosidase inhibitors. Mention will be made in particular of acarbose (Glucor®) and miglitol (Diastabol®).

Triazine derivatives with an antidiabetic effect comparable to that of metformin have been described in WO 01/55122.

Diabetic patients are moreover known to be an at-risk population as regards the development of cardiovascular pathologies, in particular arteriosclerosis and atherosclerosis. This is partly due to a greater susceptibility to factors such as hyperlipidaemia or hypercholesterolaemia. Consequently, it is recommended to maintain a low level of low-density lipoprotein (LDL) in the serum of diabetics. In particular, it will be sought to achieve this objective by means of a suitable diet and by treatments using therapeutic agents.

One particular class of compounds that are active as agents for reducing the level of LDL cholesterol in the serum is that of 3-hydroxy-3-methylglutaryl-coenzyme A (HMG-CoA) reductase inhibitors. HMG-CoA reductase inhibitors act in general on a limiting step in the regulation of cholesterol biosynthesis, and as a result reduce the total amount of cholesterol produced by the body. The compounds most commonly used in the class of HMG-CoA reductase inhibitors are the statins.

The use of statins for the treatment of diabetics has been studied. For example, U.S. Pat. No. 5,130,333 concerns a method for reducing the risk of type II diabetes (NIDDM) via administration to a patient of a hypocholesterolaemiant, such as mevastatin, lovastatin, pravastatin or velostatin.

Various combinations of compounds or various treatment methods using combinations of compounds have been developed. For example, U.S. Pat. No. 5,798,375 and U.S. Pat. No. 6,159,997 concern methods for the prevention of or treating arteriosclerosis or xanthoma via administration to a patient of a combination of HMG-CoA reductase inhibitors and of insulin sensitisers, such as thiazolidenediones. The preferred HMG-CoA reductase inhibitors are in particular pravastatin, lovastatin, simvastatin, fluvastatin, rivastatin and atorvastatin.

A treatment combining a reduction of glycaemia in parallel with a reduction of lipid factors and in particular of LDL cholesterol is thus desirable for leading to better control of the risk factors in the case of patients suffering from non-insulin-dependent diabetes and related pathologies, such as macrovascular and microvascular complications, obesity and insulin resistance.

Unexpectedly, the combinations according to the invention significantly reduce the side effects.

The applicant has developed a novel pharmaceutical composition for synergistically reducing the glycaemic and lipidic parameters of patients suffering from non-insulin-dependent diabetes, comprising the combination of an antidiabetic agent of triazine type, such as those described in WO 01/55122 and an HMG-CoA reductase inhibitor. Such a pharmaceutical composition has not been described to date.

### DESCRIPTION OF THE INVENTION

The present invention relates to a novel pharmaceutical composition comprising an HMG-CoA reductase inhibitor and a compound of the general formula (I):

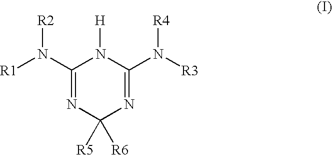

in which:
R1, R2, R3 and R4 are independently chosen from the following groups:

H,

(C1-C20)alkyl optionally substituted by halogen, (C1-C5)alkyl, (C1-C5)alkoxy or (C3-C8)cycloalkyl,

(C2-C20)alkenyl optionally substituted by halogen, (C1-C5)alkyl or (C1-C5)alkoxy

(C2-C20)alkynyl optionally substituted by halogen, (C1-C5)alkyl or (C1-C5)alkoxy

(C3-C8)cycloalkyl optionally substituted by (C1-C5)alkyl or (C1-C5)alkoxy

hetero(C3-C8)cycloalkyl bearing one or more heteroatoms chosen from N, O and S and optionally substituted by (C1-C5)alkyl or (C1-C5)alkoxy

(C6-C14)aryl(C1-C20)alkyl optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,

(C6-C14)aryl optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,

(C1-C13)heteroaryl bearing one or more heteroatoms chosen from N, O and S and optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,

R1 and R2, on the one hand, and R3 and R4, on the other hand, possibly forming with the nitrogen atom an n-membered ring (n between 3 and 8) optionally containing one or more heteroatoms chosen from N, O and S and possibly being substituted by one or more of the following groups: amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,

R5 and R6 are independently chosen from the following groups:
H,
—(C1-C20)alkyl optionally substituted by amino, hydroxyl, thio, halogen, (C1-C5)alkyl, (C1-C5)alkoxy, (C1-C5)alkylthio, (C1-C5)alkylamino, (C6-C14)-aryloxy, (C6-C14)aryl(C1-C5)alkoxy, cyano, trifluoromethyl, carboxyl, carboxymethyl or carboxyethyl,
—(C2-C20)alkenyl optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
—(C2-C20)alkynyl optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
—(C3-C8)cycloalkyl optionally substituted by amino, hydroxyl, thio, halogen, (C 1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
hetero(C3-C8)cycloalkyl bearing one or more heteroatoms chosen from N, O and S and optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
(C6-C14)aryl optionally substituted by amino, hydroxyl, thio, halogen, (C1-C5)alkyl, (C1-C5)alkoxy, (C1-C5)alkylthio, (C1-C5)alkylamino, (C6-C14)-aryloxy, (C6-C14)aryl(C1-C5)alkoxy, cyano, trifluoromethyl, carboxyl, carboxymethyl or carboxyethyl,
(C1-C13)heteroaryl bearing one or more heteroatoms chosen from N, O and S and optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
(C6-C14)aryl(C1-C5)alkyl optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
R5 and R6 possibly forming with the carbon atom to which they are attached an m-membered ring (m between 3 and 8) optionally containing one or more heteroatoms chosen from N, O and S and possibly being substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
   or possibly forming with the carbon atom a C10-C30 polycyclic residue optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl, R5 and R6 together also possibly representing the group ═O or ═S, the nitrogen atom of a heterocycloalkyl or heteroaryl group possibly being substituted by a (C1-C5)alkyl, (C3-C8)cycloalkyl, (C6-C14)aryl, (C6-C14)aryl(C1-C5)alkyl or (C1-C6)acyl group,

and also the racemic forms, tautomers, enantiomers, diastereoisomers, epimers and mixtures thereof, and the pharmaceutically acceptable salts,

and one or more pharmaceutically acceptable excipients.

The term “m-membered ring formed by R5 and R6” in particular means a saturated ring, such as a cyclohexyl, piperidyl or tetrahydropyranyl group.

The term “polycyclic group formed by R5 and R6” means an optionally substituted carbon-based polycyclic group and in particular a steroid residue.

One particular group of the invention concerns the pharmaceutical compositions according to the invention in which the triazine derivatives are compounds of the formula (I) in which R5 is hydrogen.

Another particular group of the invention concerns the pharmaceutical compositions according to the invention in which the triazine derivatives are compounds of the formula (I) in which R5 and R6 form with the carbon atom to which they are attached an m-membered ring (m between 3 and 8) optionally containing one or more heteroatoms chosen from N, O and S and possibly being substituted by one or more of the following groups: (C1-C5)alkyl, amino, hydroxyl, (C1-C5)alkylamino, alkoxy(C1-C5), (C1-C5)alkylthio, (C6-C14)aryl, (C6-C14)aryl(C1-C5)alkoxy,

or form with the carbon atom a C10-C30 polycyclic residue optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl.

Another particular group of the invention concerns the pharmaceutical compositions according to the invention in which the triazine derivatives are compounds of the formula (I) in which R5 and R6 are independently chosen from the following groups:

(C1-C20)alkyl optionally substituted by amino, hydroxyl, thio, halogen, (C1-C5)alkyl, (C1-C5)alkoxy, (C1-C5)alkylthio, (C1-C5)alkylamino, (C6-C14)-aryloxy, (C6-C14)aryl(C1-C5)alkoxy, cyano, trifluoromethyl, carboxyl, carboxymethyl or carboxyethyl.

Preferably, R1, R2, R3 and R4 are independently chosen from H and (C1-C20)alkyl groups optionally substituted by halogen, (C1-C5)alkyl, (C1-C5)alkoxy or (C3-C8)cycloalkyl; more preferably, R1=R2=H and R3=R4=(C1-C20)alkyl optionally substituted by halogen, (C1-C5)alkyl, (C1-C5)alkoxy, (C3-C8)cycloalkyl or vice versa.

Preferably, R5 and R6 are independently chosen from H and (C1-C20)alkyl groups optionally substituted by amino, hydroxyl, thio, halogen, (C1-C5)alkyl, (C1-C5)alkoxy, (C1-C5)alkylthio, (C1-C5)alkylamino, (C6-C14)-aryloxy, (C6-C14)aryl(C1-C5)alkoxy, cyano, trifluoromethyl, carboxyl, carboxymethyl or carboxyethyl; more preferably, R5=H and R6=(C1-C20)alkyl optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl or vice versa.

A more particular group of the invention concerns the pharmaceutical compositions according to the invention in which the triazine derivatives are compounds of the formula (I) in which R1 and R2 are a methyl group and R3 and R4 represent a hydrogen.

The compounds of the formula (I) may especially be chosen from:

| | Formula | Salt |
|---|---|---|
| 1 | 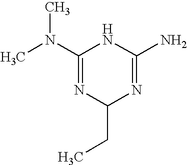 | HCl |
| 2 | 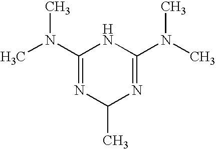 | HCl |
| 3 | 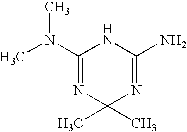 | |
| 4 | 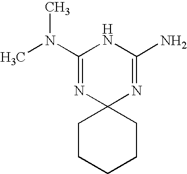 | HCl |
| 5 | 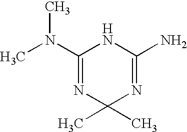 | Methane- sulfonate |
| 6 | 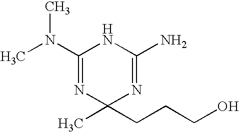 | |
| 7 | 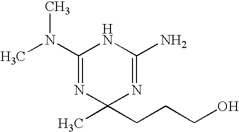 | HCl |
| 8 | 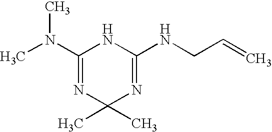 | HCl |
| 9 | 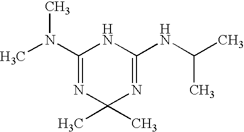 | HCl |
| 10 | 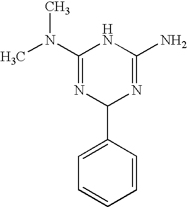 | HCl |
| 11 | 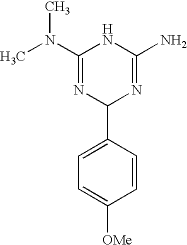 | HCl |
| 12 | 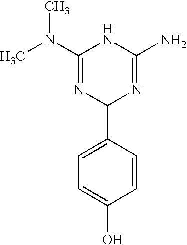 | HCl |
| 13 | 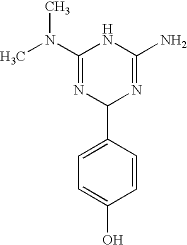 | |
| 14 | 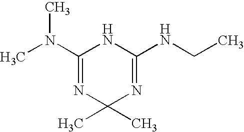 | Fumarate |
| 15 | 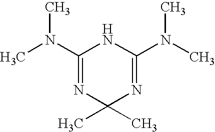 | HCl |
| 16 | 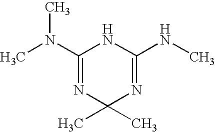 | HCl |
| 17 | 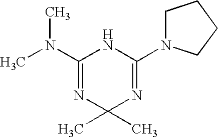 | HCl |
| 18 | 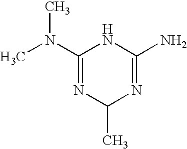 | HCl |
| 19 | 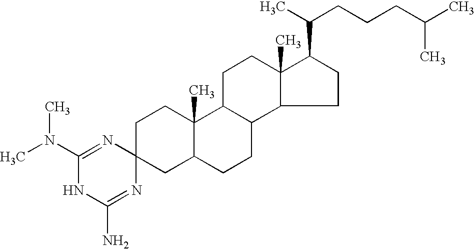 | HCl |
| 20 | 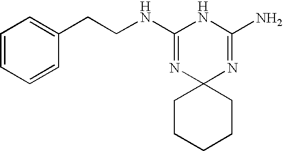 | Carbonate |
| 21 | 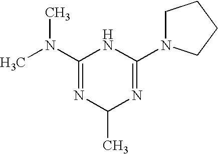 | Carbonate |
| 22 | 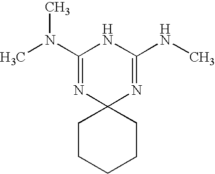 | HCl |
| 23 | 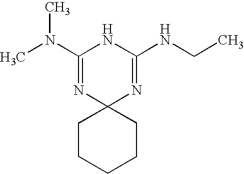 | HCl |
| 24 | 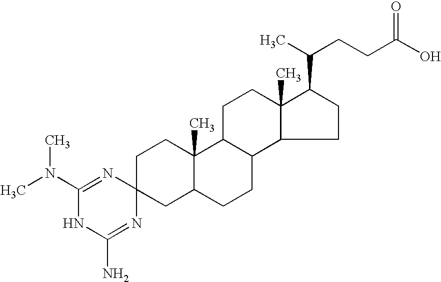 | HCl |
| 25 | 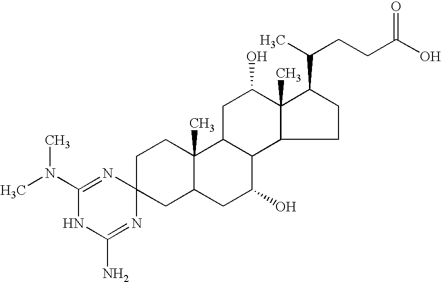 | HCl |
| 26 | 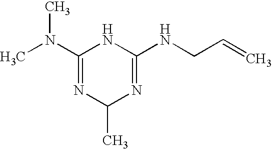 | HCl |
| 27 | 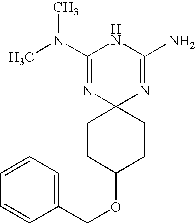 | HCl |
| 28 | 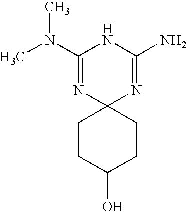 | HCl |
| 29 | 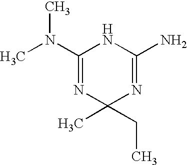 | Carbonate |
| 30 | 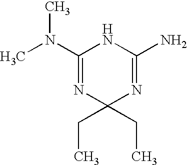 | Carbonate |
| 31 | 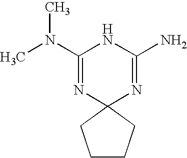 | HCl |
| 32 | 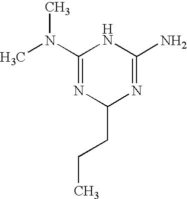 | Carbonate |
| 33 | 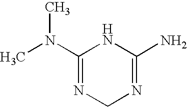 | HCl |
| 34 | 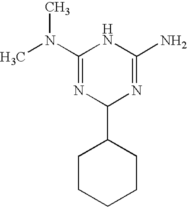 | para-Toluene- sulfonate |
| 35 | 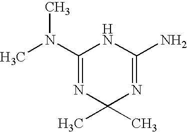 | HCl |
| 36 | 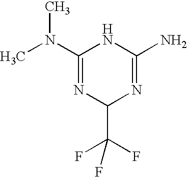 | para-Toluene- sulfonate |
| 37 | 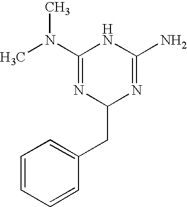 | para-Toluene- sulfonate |
| 38 | 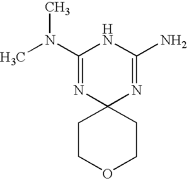 | HCl |
| 39 | 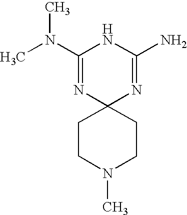 | HCl |
| 40 | 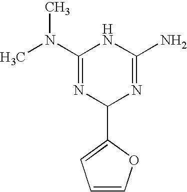 | HCl |
| 41 | 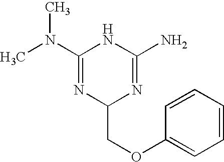 | para-Toluene- sulfonate |
| 42 | 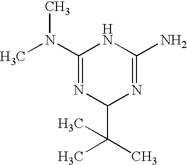 | HCl |
| 43 | 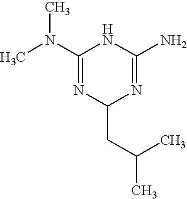 | HCl |
| 44 | 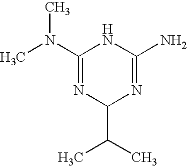 | HCl |
| 45 | 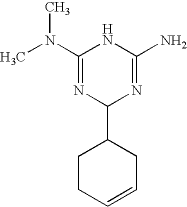 | para-Toluene- sulfonate |

and more preferably the compound of Example 18.

The term “HMG-CoA reductase inhibitor” means any HMG-CoA reductase inhibitor usually used in human or veterinary therapy.

Preferably, the HMG-CoA reductase inhibitor is a statin; more preferably, it is chosen, in a non-limiting manner, from simvastatin (Zocor®), atorvastatin (Lipitor®), fluvastatin (Lescol®), lovastatin (Mevacor®), pravastatin (Pravachol®), rosuvastatin (Crestor®), velostatin, itavastatin, synvinolin and pitivastatin. The statins may also be in the form of pharmaceutically acceptable salts, such as, in a non-limiting manner, the hydrochloride, hydrobromide, hydroiodide, sulfate, nitrate, phosphate, citrate, methanesulfonate, trifluoroacetate or acetate, the sodium ion, the potassium ion, the calcium ion or the magnesium ion.

According to yet another preferred embodiment, the invention relates more particularly to the pharmaceutical compositions chosen from:
(+)-2-amino-3,6-dihydro-4-dimethylamino-6-methyl-1,3,5-triazine hydrochloride and simvastatin
(+)-2-amino-3,6-dihydro-4-dimethylamino-6-methyl-1,3,5-triazine hydrochloride and atorvastatin
(+)-2-amino-3,6-dihydro-4-dimethylamino-6-methyl-1,3,5-triazine hydrochloride and fluvastatin
(+)-2-amino-3,6-dihydro-4-dimethylamino-6-methyl-1,3,5-triazine hydrochloride and lovastatin
(+)-2-amino-3,6-dihydro-4-dimethylamino-6-methyl-1,3,5-triazine hydrochloride and pravastatin
(+)-2-amino-3,6-dihydro-4-dimethylamino-6-methyl-1,3,5-triazine hydrochloride and rosuvastatin.

The invention also relates to the racemic forms, tautomers, enantiomers, diastereoisomers and epimers, and mixtures thereof, of the compounds of the general formula (I).

The compounds of the invention of the formula (I) as defined above, containing a sufficiently basic function, or both, may include the corresponding pharmaceutically acceptable salts of organic or mineral acids.

For the purposes of the present invention, the term “corresponding pharmaceutically acceptable salts of organic or mineral acids” means any salt prepared from any non-toxic pharmaceutically acceptable organic or inorganic acid. Such acids include acetic acid, benzenesulfonic acid, benzoic acid, citric acid, carbonic acid, ethanesulfonic acid, fumaric acid, gluconic acid, glutamic acid, hydrobromic acid, hydrochloric acid, lactic acid, mandelic acid, malic acid, maleic acid, methanesulfonic acid, mucic acid, nitric acid, pamoic acid, pantothenic acid, phosphoric acid, succinic acid, tartaric acid and para-toluenesulfonic acid. Hydrochloric acid is advantageously used.

The invention also relates to the chiral salts of the compounds of the formula (I) used for the separation of the racemates of the compounds of the formula (I).

By way of example, the following chiral acids are used: (+)-D-di-O-benzoyltartaric acid, (−)-L-di-O-benzoyltartaric acid, (−)-L-di-O,O′-p-toluoyl-L-tartaric acid, (+)-D-di-O,O′-p-toluyl-L-tartaric acid, (R)-(+)-malic acid, (S)-(−)-malic acid, (+)-camphanic acid, (−)-camphanic acid, R-(−)-1,1′-binaphthalen-2,2′-diylhydrogenophosphonic acid, (+)-camphoric acid, (−)-camphoric acid, (S)-(+)-2-phenylpropionic acid, (R)-(+)-2-phenylpropionic acid, D-(−)-mandelic acid, L-(+)-mandelic acid, D-tartaric acid, L-tartaric acid, or a mixture of two or more thereof.

The compounds of the formula (I) above also include the prodrugs of these compounds.

The term “prodrugs” means compounds which, when administered to the patient, are chemically and/or biologically converted in the live body into compounds of the formula (I).

It will be appreciated that the compounds that are useful according to the present invention may contain asymmetric centres. These asymmetric centres may be, independently, in R or S configuration. It will be clear to a person skilled in the art that certain compounds that are useful according to the invention may also exhibit geometrical isomerism. It should be understood that the present invention includes individual geometrical isomers and stereoisomers and mixtures thereof, including racemic mixtures, of compounds of the formula (I) above. Isomers of this type can be separated from mixtures thereof by application or adaptation of known processes, for example chromatography techniques or recrystallisation techniques, or they are prepared separately from suitable isomers of their intermediates.

The enantiomers of the compounds according to the invention and the process for the preparation of them are especially described in patent application WO 2004/089917, the content of which is incorporated herein by reference.

The present patent application also concerns the polymorphic forms of the compounds, as obtained according to patent application WO 2004/089917, for instance the A1 polymorphic form of the salt (+)-2-amino-3,6-dihydro-4-dimethylamino-6-methyl-1,3,5-triazine hydrochloride.

The present invention also relates to the other polymorphic forms of the compounds, such as the H1 polymorphic form of the salt (+)-2-amino-3,6-dihydro-4-dimethylamino-6-methyl-1,3,5-triazine hydrochloride, which can be prepared as follows:

Approximately 3 g of the A1 form of Example 18 are dissolved in 50 ml of 1 mol/l HCl at room temperature. The clear solution obtained is left to evaporate at room temperature, in an open beaker, until a solid residue crystallises. The characterisation is performed by:

FT-IR spectroscopy:
Brüker Vector 22
2 cm⁻¹ spectral resolution
32 scans
KBR discs (analogous to method A AA21505)
To evaluate the intensity of the IR bands, the IR spectra were normalised by vectorisation in the spectral range 4000-400 cm⁻¹ as an absorption spectrum.

Preadjustment was performed:
s: A>0.05
m: 0.01<A<0.05
w: A<0.01.

FT-Raman spectroscopy:
Brüker RFS-100
excitation: 1064 nm
spectral resolution: 1 cm⁻¹
1000 mW
1000 scans
focalised
aluminium crucible (analogous to method RA AA21505)
To evaluate the intensity of the Raman bands, Raman spectra were normalised by vectorisation in the spectral range 3600-200 cm⁻¹. Pre-adjustment was performed:
   s: A>0.05
   m: 0.01<A<0.05
   w: A<0.01

Powder x-ray diffraction (XRD)
diffractometer D5000 (Balker AXS)
radiation CuKα1 at 1.5406 Å (U=30 kV, A=40 mA)
Transmission mode
Detector in sensitive position
Primary monochromator
Angle range: 3-65° 2θ
Stage width: 0.05° 2θ
Measuring time/stage: 1.4 s
The XRD machine is set at 2θ±0.1°.

### Results

### A1 Form:

### XRD:

| No. | d[Å] | 2θ | I/Io |
|---|---|---|---|
| 1 | 5.98 | 14.8 | 85 |
| 2 | 5.26 | 16.8 | 83 |
| 3 | 4.35 | 20.4 | 30 |
| 4 | 3.57 | 24.9 | 100 |
| 5 | 3.50 | 25.4 | 53 |
| 6 | 3.36 | 26.5 | 96 |
| 7 | 3.31 | 26.9 | 52 |
| 8 | 3.04 | 29.3 | 57 |
| 9 | 2.90 | 30.8 | 30 |
| 10 | 2.74 | 32.7 | 35 |

FT-IR bands (in cm⁻¹): 3384+/−1.5 (m), 3199+/−1.5 (m), 3163+/−1.5 (m), 3107+/−1.5 (m), 2993+/−1.5 (m), 2983+/−1.5 (m), 1652+/−1.5 (s), 1606+/−1.5 (s), 1576+/−1.5 (s), 1557+/−1.5 (s), 1505+/−1.5 (s), 1449+/−1.5 (m), 1427+/−1.5 (m), 1405+/−1.5 (m), 1383+/−1.5 (m), 1348+/−1.5 (m), 1306+/−1.5 (m), 1263+/−1.5 (w), 1235+/−1.5 (w), 1185+/−1.5 (w), 1096+/−1.5 (w), 1068+/−1.5 (w), 980+/−1.5 (w), 946+/−1.5 (w), 868+/−1.5 (w), 761+/−1.5 (w), 687+/−1.5 (m), 655+/−1.5 (m), 558+/−1.5 (w), 521+/−1.5 (w), 478+/−1.5 (w)
FT-Raman bands (in cm⁻¹): 3217+/−1.5 (w), 2994+/−1.5 (m), 2983+/−1.5 (m), 2936+/−1.5 (s), 2883+/−1.5 (m), 1645+/−1.5 (w), 1602+/−1.5 (m), 1554+/−1.5 (m), 1453+/−1.5 (m), 1428+/−1.5 (m), 1349+/−1.5 (w), 1308+/−1.5 (w), 979+/−1.5 (m), 866+/−1.5 (w), 761+/−1.5 (w), 686+/−1.5 (s), 583+/−1.5 (m), 555+/−1.5 (s), 525+/−1.5 (m), 479+/−1.5 (m), 410+/−1.5 (m), 401+/−1.5 (m), 307+/−1.5 (m) H1 form

### XRD:

| No. | d[Å] | 2θ | I/Io |
|---|---|---|---|
| 1 | 8.03 | 11.0 | 69 |
| 2 | 7.27 | 12.2 | 25 |
| 3 | 6.11 | 14.5 | 24 |
| 4 | 4.01 | 22.1 | 86 |
| 5 | 3.64 | 24.5 | 100 |
| 6 | 3.26 | 27.3 | 51 |
| 7 | 3.08 | 29.0 | 29 |
| 8 | 3.04 | 29.4 | 34 |
| 9 | 2.82 | 31.7 | 61 |
| 10 | 2.66 | 33.6 | 26 |

FT-IR bands (in cm⁻¹): 3386+/−1.5 (m), 3080+/−3 (m), 1706+/−1.5 (s), 1691+/−1.5 (s), 1634+/−1.5 (m), 1513+/−1.5 (m), 1445+/−1.5 (w), 1241+/−1.5 (w), 1079+/−1.5 (w), 989+/−1.5 (w), 940+/−1.5 (w), 861+/−1.5 (w), 823+/−1.5 (w), 675+/−1.5 (w), 603+/−1.5 (w), 573+/−1.5 (w), 549+/−1.5 (w), 527+/−1.5 (w)

For the purposes of this text, it is understood that the tautomeric forms are included in the mention of a given group, for example thio/mercapto or oxo/hydroxy.

In the present description, the terms used have, unless otherwise indicated, the following meanings:
the term “(C1-C20)alkyl” denotes a linear or branched alkyl radical containing from 1 to 20 carbon atoms. Among the C1-C20 alkyl radicals that may especially be mentioned, in a non-limiting manner, are methyl, ethyl, propyl, isopropyl, butyl, sec-butyl, tert-butyl, pentyl, hexyl, octyl, decyl, dodecyl, hexadecyl and octadecyl radicals;
the term “(C1-C20)alkenyl” denotes a linear or branched hydrocarbon-based radical containing one or more unsaturations in double bond form. As alkylene radicals containing from 1 to 20 carbon atoms, mention may be made, in a non-limiting manner, of ethenyl, prop-2-enyl, but-2-enyl, but-3-enyl, pent-2-enyl, pent-3-enyl and pent-4-enyl radicals;
the term “(C1-C20)alkynyl” denotes a linear or branched hydrocarbon-based radical containing one or more unsaturations in triple bond form. As alkylene radicals containing from 1 to 20 carbon atoms, mention may be made, in a non-limiting manner, of ethynyl, prop-2-ynyl, but-2-ynyl, but-3-ynyl, pent-2-ynyl, pent-3-ynyl and pent-4-ynyl radicals;
the term “alkoxy” refers to the term “alkyl-oxy”;
the term “halogen” refers, in a non-limiting manner, to fluorine, chlorine or bromine;
the term “(C6-C14)aryl” refers to an aromatic group containing from 6 to 14 carbon atoms with at least one of the rings having a system of conjugated pi electrons, and including biaryls, which may be optionally substituted. Mention will be made in particular of biphenyl, phenyl, naphthyl, anthryl and phenanthryl radicals;
the term “(C6-C14)aryl(C1-C20)alkyl” refers to the corresponding-alkylaryl groups. Mention will be made in particular of benzyl and phenethyl groups;
the term “hetero(C6-C14)aryl” refers to a 6-14-membered aromatic heterocycle containing 1-4 heteroatoms, the other atoms being carbon atoms. Among the heteroatoms, mention will be made in particular of oxygen, sulfur and nitrogen. Among the heteroaryl radicals, mention will be made more particularly of furyl, thienyl, pyridyl, pyrrolyl, pyrimidyl, pyrazinyl, oxazolyl, oxadiazolyl, isoxazolyl, quinolyl and thiazolyl radicals;
the term “(C3-C8)cycloalkyl” refers to a saturated hydrocarbon-based ring and contains monocyclic, bicyclic and polycyclic radicals containing from 3 to 8 carbon atoms. Mention will be made, in a non-limiting manner, of cyclopropyl and cyclobutyl radicals.

The pharmaceutical compositions according to the present invention are useful in the treatment of pathologies associated with insulin resistance syndrome (syndrome X).

Insulin resistance is characterised by a reduction in the action of insulin (cf. Presse Médicale, 1997, 26 (No. 14), 671-677) and is involved in a large number of pathological conditions, such as diabetes and more particularly non-insulin-dependent diabetes (type II diabetes or NIDDM), dyslipidaemia, obesity and arterial hypertension, and also certain microvascular and macrovascular complications, for instance atherosclerosis, retinopathy and neuropathy.

In this respect, reference will be made, for example, to Diabetes, vol. 37, 1988, 1595-1607; Journal of Diabetes and its Complications, 1998, 12, 110-119 or Horm. Res., 1992, 38, 28-32.

The aim of the present invention is to propose a pharmaceutical composition for significantly improving the condition of diabetics.

The pharmaceutical compositions of the invention especially have hypoglycaemiant activity.

The compounds of the formula (I) are therefore useful in the treatment of pathologies associated with hyperglycaemia.

The pharmaceutical composition comprising the triazine compound of the formula (I) in combination with a statin can be prepared by mixing together the various active principles, either all together or independently with a physiologically acceptable support, an excipient, a binder, a diluent, etc. It is then administered orally or non-orally, for instance via the parenteral, intravenous, cutaneous, nasal or rectal route. If the active principles are formulated independently, the corresponding formulations can be mixed together extemporaneously using a diluent and are then administered or can be administered independently of each other, either successively or sequentially.

The pharmaceutical compositions of the invention include formulations such as granules, powders, tablets, gel capsules, syrups, emulsions and suspensions, and also forms used for non-oral administration, for instance injections, sprays or suppositories.

The pharmaceutical forms can be prepared via the known conventional techniques.

The preparation of an orally administered solid pharmaceutical form will be performed by the following process: an excipient (for example lactose, sucrose, starch, mannitol, etc.), a disintegrant (for example calcium carbonate, calcium carboxymethylcellulose, alginic acid, sodium carboxymethylcellulose, colloidal silicon dioxide, sodium croscarmellose, Crospovidone, guar gum, magnesium aluminium silicate, microcrystalline cellulose, cellulose powder, pregelatinised starch, sodium alginate, starch glycolate, etc.), a binder (for example alpha-starch, gum arabic, carboxymethylcellulose, polyvinylpyrrolidone, hydroxypropylcellulose, alginic acid, carbomer, dextrin, ethylcellulose, sodium alginate, maltodextrin, liquid glucose, magnesium aluminium silicate, hydroxyethylcellulose, methylcellulose, guar gum, etc.) and a lubricant (for example talc, magnesium stearate, polyethylene 6000, etc.) are, for example, added to the active principle(s) and the mixture obtained is then tabletted. If necessary, the tablet can be coated via the known techniques, in order to mask the taste (for example with cocoa powder, mint, borneol, cinnamon powder, etc.) or to allow enteric dissolution or sustained release of the active principles. The coating products that can be used are, for example, ethylcellulose, hydroxymethylcellulose, polyoxyethylene glycol, cellulose acetophthalate, hydroxypropylmethylcellulose phthalate and Eudragit® (methacrylic acid-acrylic acid copolymer), Opadry® (hydroxypropylmethylcellulose+macrogol+titanium oxide+lactose monohydrate). Pharmaceutically acceptable colorants may be added (for example yellow iron oxide, red iron oxide, quinoline yellow lake, etc.). Pharmaceutical forms such as tablets, powders, sachets and gel capsules can be used for an oral administration.

The liquid pharmaceutical forms for oral administration include solutions, suspensions and emulsions. The aqueous solutions can be obtained by dissolving the active principles in water, followed by addition of flavourings, colorants, stabilisers and thickener, if necessary. In order to improve the solubility, it is possible to add ethanol, propylene glycol or other pharmaceutically acceptable non-aqueous solvents. The aqueous suspensions for oral use can be obtained by dispersing the finely divided active principles in water with a viscous product, such as natural or synthetic gums, resins, methylcellulose or sodium carboxymethylcellulose.

The pharmaceutical forms for injection can be obtained, for example, by the following process. The active principle(s) is (are) dissolved, suspended or emulsified either in an aqueous medium (for example distilled water, physiological saline, Ringer's solution, etc.) or in an oily medium (for example a plant oil, such as olive oil, sesameseed oil, cottonseed oil, corn oil, etc., or propylene glycol), with a dispersant (for example Tween 80, HCO 60 (Nikko Chemicals), polyethylene glycol, carboxymethylcellulose, sodium alginate, etc.), a preserving agent (for example methyl p-hydroxybenzoate, propyl p-hydroxybenzoate, benzyl alcohol, chlorobutanol, phenol, etc.), an isotonicity agent (for example sodium chloride, glycerol, sorbitol, glucose, etc.) and also other additives, such as, if desired, a solubilising agent (for example sodium salicylate, sodium acetate, etc.) or a stabiliser (for example human serum albumin).

A pharmaceutical form for external use can be obtained from a solid, semi-solid or liquid composition containing the active principle(s). For example, to obtain a solid form, the active principle(s) is (are) treated, alone or as mixtures, with excipients (for example lactose, mannitol, starch, microcrystalline cellulose, sucrose, etc.) and a thickener (for example natural gums, cellulose derivatives, acrylic polymers, etc.) so as to convert them into powder. The liquid pharmaceutical compositions are prepared in substantially the same way as the forms for injection, as indicated previously. The semi-solid pharmaceutical forms are preferably in the form of aqueous or oily gels or in the form of a pomade. These compositions may optionally contain a pH regulator (for example carbonic acid, phosphoric acid, citric acid, hydrochloric acid, sodium hydroxide, etc.) and a preserving agent (for example p-hydroxybenzoic acid esters, chlorobutanol, benzalkonium chloride, etc.) and also other additives.

If, in the present patent application, lovastatin is used, the daily dose is between 10 mg and 40 mg, more preferably 20 mg. If fluvastatin is used, the daily dose is between 20 mg and 40 mg. If atorvastatin is used, the daily dose is between 10 mg and 80 mg and preferably between 10 mg and 40 mg. If simvastatin is used, the daily dose is between 5 mg and 50 mg and preferably between 5 mg and 20 mg. If cerivastatin is used, the daily dose is between 0.1 mg and 0.8 mg and preferably between 0.1 mg and 0.3 mg. If pravastatin is used, the daily dose is between 10 mg and 40 mg, preferably 20 mg. If atavastatin is used, the daily dose is between 1 mg and 20 mg and preferably between 2 mg and 20 mg. If rosuvastatin is used, the daily dose is between 4 mg and 80 mg and preferably between 10 mg and 20 mg.

The daily doses of the compounds of the formula (I) are between 200 mg and 2000 mg.

The relative proportion of the constituents of the pharmaceutical compositions of the present invention takes into account the recommended dosages of the respective active principles. These relative proportions of HMG-CoA reductase inhibitors, or of pharmaceutically acceptable salts thereof, and of the compounds of the formula (I), or of pharmaceutically acceptable salts thereof, thus vary in consequence. Preferably, the weight ratio of the HMG-CoA reductase inhibitor to the compound of the formula (I) ranges between ½ and 1/20 000, more particularly from ¼ to 1/2000 and especially from ⅕ to 1/2000. The frequency of administration of the compounds of the invention is between 1 and 2 administrations per day. In cases where the doses of compounds of the formula (I) necessitate more than one daily administration, the amounts of HMG-CoA reductase inhibitors and the HMG-CoA reductase inhibitor/compound of the formula (I) ratios will be adjusted in consequence.

The aim of the present invention is also to propose a method of treatment via co-administration of effective doses of a compound of the formula (I) and of an HMG-CoA reductase inhibitor, and also kits for allowing this co-administration.

The present invention also relates to kits that are suitable for the treatment by the methods described above. These kits comprise a composition containing the compound of the formula (I) in the dosages indicated above and a second composition containing the HMG-CoA reductase inhibitors in the dosages indicated above, for a simultaneous, separate or sequential administration, in effective amounts according to the invention.

The term “co-administration” means the simultaneous, separate or sequential administration of one or more compounds to the same patient, over a period that may be up to 2 hours or even up to 12 hours. For example, the term co-administration includes:

(1) a simultaneous administration of the two compounds,

(2) an administration of the first, followed 2 hours later by the administration of the second compound,

(3) an administration of the first, followed 12 hours later by the administration of the second compound.

The examples below of compositions according to the invention are given as non-limiting illustrations.

### EXAMPLES

The amounts are expressed on a weight basis.

### Formulation Example 1

### (+)-2-amino-3,6-dihydro-4-dimethylamino-6-methyl-1,3,5-triazine hydro-

chloride: 1000 mg

atorvastatin: 10 mg

microcrystalline cellulose: 110 mg

croscarmellose: 28 mg

polyvinylpyrrolidone: 40 mg

magnesium stearate: 14 mg

Opadry: 24 mg

### Formulation Example 2

### (+)-2-amino-3,6-dihydro-4-dimethylamino-6-methyl-1,3,5-triazine hydro-

chloride: 1000 mg

fluvastatin: 20 mg

microcrystalline cellulose: 115.5 mg

croscarmellose: 28 mg

polyvinylpyrrolidone: 40 mg

magnesium stearate: 9 mg

Opadry®: 24 mg

### Formulation Example 3

### (+)-2-amino-3,6-dihydro-4-dimethylamino-6-methyl-1,3,5-triazine hydro-

chloride: 750 mg

pravastatin: 10 mg

microcrystalline cellulose: 89 mg

croscarmellose: 21 mg

polyvinylpyrrolidone: 30 mg

magnesium stearate: 10.5 mg

Opadry®: 18 mg

### Formulation Example 4

### (+)-2-amino-3,6-dihydro-4-dimethylamino-6-methyl-1,3,5-triazine hydro-

chloride: 1000 mg

atorvastatin: 30 mg

microcrystalline cellulose: 150 mg

croscarmellose: 24 mg

polyvinylpyrrolidone: 44 mg

magnesium stearate: 8 mg

Eudragit®: 24 mg

### Formulation Example 5

### (+)-2-amino-3,6-dihydro-4-dimethylamino-6-methyl-1,3,5-triazine hydro-

chloride: 1000 mg

lovastatin: 20 mg

Silicon dioxide: 4 mg

croscarmellose: 25 mg

polyvinylpyrrolidone: 40 mg

magnesium stearate: 8 mg

Opadry®: 10 mg

Biological Results for the Combinations According to the Invention

The synergistic action of the combinations according to the invention is demonstrated using an animal model. Obese rats (obese Zucker (fa/fa)) are used to simulate non-insulin-dependent diabetes (NIDDM).

The action of lovastatin alone and of the compound (+)-2-amino-3,6-dihydro-4-dimethylamino-6-methyl-1,3,5-triazine, hydrochloride salt, alone and the combination of these two agents is evaluated in terms of triglycerides, total cholesterol, high-density lipoprotein C (HDL C), glucose and insulin. The rats is received the treatment for 5 consecutive days. The blood samples are collected 3 days before and 5 days after the start of the treatments in order to measure the levels of triglycerides, total cholesterol, HDL C, glucose and insulin.

The following procedure is adopted.

Four groups of eight rats are formed:
a “vehicle” group;
a group that receives a dose of 1 mg/kg/day of lovastatin orally;
a group that receives a dose of 50 mg/kg or 100 mg/kg twice a day (bid) of (+)-2-amino-3,6-dihydro-4-dimethylamino-6-methyl-1,3,5-triazine, hydrochloride salt, orally;
a group that receives a dose of 1 mg/kg/day of lovastatin+50 mg/kg or 100 mg/kg twice a day (bid) of (+)-2-amino-3,6-dihydro-4-dimethylamino-6-methyl-1,3,5-triazine, hydrochloride salt, orally.

The statistical analyses consist of an analysis of variance to a classification criterion, followed by multiple comparisons versus the vehicle group (Dunnett test). To evaluate the meaning of the results obtained, the values are expressed as a mean±SEM. A difference is considered significant for p<0.05. The results are expressed as millimol per litre (mM) or nanomol per litre (nM).

1. Pharmaceutical composition comprising, as active principle:
i) an HMG-CoA reductase inhibitor,
ii) a triazine derivative of the formula (I) 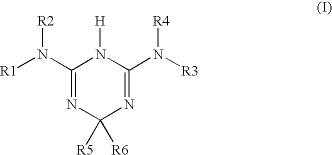
in which:
R1, R2, R3 and R4 are independently chosen from the following groups:
H,
(C1-C20)alkyl optionally substituted by halogen, (C1-C5)alkyl, (C1-C5)alkoxy or (C3-C8)cycloalkyl,
(C2-C20)alkenyl optionally substituted by halogen, (C1-C5)alkyl or (C1-C5)alkoxy
(C2-C20)alkynyl optionally substituted by halogen, (C1-C5)alkyl or (C1-C5)alkoxy
(C3-C8)cycloalkyl optionally substituted by (C1-C5)alkyl or (C1-C5)alkoxy
hetero(C3-C8)cycloalkyl bearing one or more heteroatoms chosen from N, O and S and optionally substituted by (C1-C5)alkyl or (C1-C5)alkoxy
(C6-C14)aryl(C1-C20)alkyl optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
(C6-C14)aryl optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
(C1-C13)heteroaryl bearing one or more heteroatoms chosen from N, O and S and optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
R1 and R2, on the one hand, and R3 and R4, on the other hand, possibly forming with the nitrogen atom an n-membered ring (n between 3 and 8) optionally containing one or more heteroatoms chosen from N, O and S and possibly being substituted by one or more of the following groups: amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
R5 and R6 are independently chosen from the following groups:
H,
(C1-C20)alkyl optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
(C2-C20)alkenyl optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
(C2-C20)alkynyl optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
(C3-C8)cycloalkyl optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
hetero(C3-C8)cycloalkyl bearing one or more heteroatoms chosen from N, O and S and optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
(C6-C14)aryl optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
(C1-C13)heteroaryl bearing one or more heteroatoms chosen from N, O and S and optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
(C6-C14)aryl(C1-C5)alkyl optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
R5 and R6 possibly forming with the carbon atom to which they are attached an m-membered ring (m between 3 and 8) optionally containing one or more heteroatoms chosen from N, O and S and possibly being substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
or possibly forming with the carbon atom a C10-C30 polycyclic residue optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl,
R5 and R6 together also possibly representing the group ═O or ═S, the nitrogen atom of a heterocycloalkyl or heteroaryl group possibly being substituted by a (C1-C5)alkyl, (C3-C8)cycloalkyl, (C6-C14)aryl, (C6-C14)aryl(C1-C5)alkyl or (C1-C6)acyl group,
and also the racemic forms, tautomers, enantiomers, diastereoisomers, epimers and polymorphs, and mixtures thereof, and the pharmaceutically acceptable salts,
and one or more pharmaceutically acceptable excipients.

2. Pharmaceutical composition according to claim 1, comprising a compound of the formula (I) in which R5 is hydrogen.

3. Pharmaceutical composition according to claim 1, comprising a compound of the formula (I) in which R5 and R6 are independently chosen from H and (C1-C20)alkyl groups optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxyl, carboxymethyl or carboxyethyl.

4. Pharmaceutical composition according to claim 1, comprising a compound of the formula (I) in which R1, R2, R3 and R4 are independently chosen from H and (C1-C20)alkyl groups optionally substituted by halogen, (C1-C5)alkyl, (C1-C5)alkoxy or (C3-C8)cycloalkyl.

5. Pharmaceutical composition according to claim 1, comprising a compound of the formula (I) in which R5 and R6 are independently chosen from H and (C1-C20)alkyl groups optionally substituted by amino, hydroxyl, thio, halogen, (C1-C5)alkyl, (C1-C5)alkoxy, (C1-C5)alkylthio, (C1-C5)alkylamino, (C6-C14)aryloxy, (C6-C14)aryl(C1-C5)alkoxy, cyano, trifluoromethyl, carboxy, carboxymethyl or carboxyethyl.

6. Pharmaceutical composition according to claim 1, comprising a compound of the formula (I) in which R1 and R2 are a methyl group and R3 and R4 represent a hydrogen.

7. Pharmaceutical composition according to claim 1, characterised in that the compound of the formula (I) is 2-amino-3,6-dihydro-4-dimethylamino-6-methyl-1,3,5-triazine.

8. Pharmaceutical composition according to claim 1, characterised in that the compound of the formula (I) is (−)-2-amino-3,6-dihydro-4-dimethylamino-6-methyl-1,3,5-triazine.

9. Pharmaceutical composition according to claim 1, characterised in that the compound of the formula (I) is (+)-2-amino-3,6-dihydro-4-dimethylamino-6-methyl-1,3,5-triazine.

10. Pharmaceutical composition according to claim 1, characterised in that the compound of the formula (I) is in the form of a hydrochloride.

11. Pharmaceutical composition according to claim 1, such that the HMG-CoA reductase inhibitor is a statin.

12. Pharmaceutical composition according to claim 11, characterised in that the statins are in the form of a salt chosen from the hydrochloride, hydrobromide, hydroiodide, sulfate, nitrate, phosphate, citrate, methanesulfonate, trifluoroacetate and acetate, the sodium ion, the potassium ion, the calcium ion and the magnesium ion.

13. Pharmaceutical composition according to claim 1, characterised in that these pharmaceutical compositions contain between 0.1 mg and 80 mg of HMG-CoA reductase inhibitor.

14. Pharmaceutical composition according to claim 1, characterised in that these pharmaceutical compositions contain between 200 mg and 2000 mg of a compound of the formula (I).

15. Pharmaceutical composition according to claim 1, characterised in that the weight ratio of the HMG-CoA reductase inhibitor to the compound of the formula (I) is between ½ and 1/20 000.

16. Pharmaceutical composition according to claim 1, characterised in that the statin is chosen from simvastatin, atorvastatin, fluvastatin, lovastatin, pravastatin, rosuvastatin, velostatin, itavastatin, synvinolin and pitivastatin.

17. Pharmaceutical composition according to claim 1, characterised in that the statin is simvastatin, atorvastatin, fluvastatin, lovastatin, pravastatin or rosuvastatin.

18. Pharmaceutical composition according to claim 1, characterised in that the statin is simvastatin and the compound of the formula (I) is (+)-2-amino-3,6-dihydro-4-dimethylamino-6-methyl-1,3,5-triazine, optionally in the form of a hydrochloride.

19. Pharmaceutical composition according to claim 1, characterised in that the statin is atorvastatin and the compound of the formula (I) is (+)-2-amino-3,6-dihydro-4-dimethylamino-6-methyl-1,3,5-triazine, optionally in the form of a hydrochloride.

20. Pharmaceutical composition according to claim 1, characterised in that the statin is fluvastatin and the compound of the formula (I) is (+)-2-amino-3,6-dihydro-4-dimethylamino-6-methyl-1,3,5-triazine, optionally in the form of a hydrochloride.

21. Pharmaceutical composition according to claim 1, characterised in that the statin is lovastatin and the compound of the formula (I) is (+)-2-amino-3,6-dihydro-4-dimethylamino-6-methyl-1,3,5-triazine, optionally in the form of a hydrochloride.

22. Pharmaceutical composition according to claim 1, characterised in that the statin is pravastatin and the compound of the formula (I) is (+)-2-amino-3,6-dihydro-4-dimethylamino-6-methyl-1,3,5-triazine, optionally in the form of a hydrochloride.

23. Pharmaceutical composition according to claim 1, characterised in that the statin is rosuvastatin and the triazine derivative is (+)-2-amino-3,6-dihydro-4-dimethylamino-6-methyl-1,3,5-triazine, optionally in the form of a hydrochloride.

24. Pharmaceutical composition according to claim 1, which is suitable for oral administration, in which the pharmaceutical composition is a powder, a coated tablet, a gel capsule, a sachet, a solution, a suspension or an emulsion.

25. A method for the treatment of/and/or preventing diabetes comprising administering an HMG-CoA reductase inhibitor in combination with a compound of the formula (I) as defined in claim 1.

26. A method of claim 25 for the treatment of and/or preventing non-insulin-dependent diabetes.

27. A method for the treatment of at least one of the pathologies associated with insulin resistance syndrome, chosen from dyslipidaemia, obesity, arterial hypertension, and microvascular and macrovascular complications, for instance atherosclerosis, retinopathy, nephropathy and neuropathy comprising administering an HMG-CoA reductase inhibitor in combination with a compound of the formula (I) as defined in claim 1.

28. Use wherein the combination is defined in claim 16.

29. Use wherein the combination is as defined in claim 18.

30. Method according to claim 25, such that the administration of compound (I) and that of the HMG-CoA reductase inhibitor are simultaneous, separate or sequential.

31. Kit comprising a compound of the formula (I) as defined according to and an HMG-CoA reductase inhibitor as defined according to claim 16, for simultaneous, separate or sequential administration.
